(19) United States
(12) Patent Application Publication
(10) Pub. No.: US 2025/0375162 A1
(43) Pub. Date: Dec. 11, 2025

(54) MULTI-FACETED IMPLANTABLE PHYSIOLOGICAL INTERFACES

(71) Applicant: NEUFLUENT LLC, Seattle, WA (US)

(72) Inventors: Samuel Robert Browd, Mercer Island, WA (US); Ryan J. Douglas, Seattle, WA (US)

(73) Assignee: NEUFLUENT LLC, Seattle, WA (US)

(21) Appl. No.: 19/230,460

(22) Filed: Jun. 6, 2025

Related U.S. Application Data
(60) Provisional application No. 63/657,489, filed on Jun. 7, 2024.

(51) Int. Cl. A61B 5/00

(52) U.S. Cl. CPC A61B 5/686; A61B 5/0031; A61B 5/4836; A61B 5/6885; A61B 5/7203; A61B 5/746; A61B 2560/0214; A61B 2560/0223; A61B 2560/0406; A61B 2560/0468; A61B 2560/063; A61B 2562/046

(57) ABSTRACT

Apparatus and associated methods relate to multi-faceted implantable physiological interface system including an array of panels each panel including a substrate defining opposing first and second sides, a bioprocessor supported on the first side and operatively coupled to an electrode disposed on the second side. In an illustrative example, the substrate of each of the panels joins the substrate of at least one other adjacent panel such that the array of panels collectively define an outer enclosure having an internal volume configured to house one or more internal components. The internal components, may, for example, include sensors, actuators, therapeutic components, communication modules, data stores, security modules, thermal management units, and power sources. Various embodiments may advantageously provide multi-faceted, modular, adaptive physiological interfaces enabling precise monitoring, stimulation, and therapeutic interventions while dynamically conforming to a target environment.

### CROSS-REFERENCE TO RELATED APPLICATIONS

This application is a non-provisional application and claims the benefit of U.S. Application Ser. No. 63/657,489, titled “MULTI-FACETED IMPLANTABLE PHYSIOLOGICAL INTERFACES,” filed by Samuel Robert Browd et al., on Jun. 7, 2024.

This application incorporates the entire contents of the foregoing application(s) herein by reference.

### TECHNICAL FIELD

Various embodiments relate generally to an implantable physiological device, particularly, for example, to monitor and/or treat neurological conditions.

### BACKGROUND

Neurological diseases encompass a broad spectrum of disorders affecting the brain, spinal cord, and nerves, leading to impairments in movement, cognition, and overall neurological function. These diseases include neurodegenerative conditions such as Alzheimer's and Parkinson's, autoimmune disorders like multiple sclerosis, and acute conditions such as strokes. Given their complexity, neurological diseases often have multifaceted treatment approaches aimed at slowing disease progression, alleviating symptoms, and improving patients' quality of life.

Brain-Computer Interface (BCI) technology represents an evolving field focused on enabling direct communication between the brain and external devices. BCIs may, for example, record and interpret neural signals, such as electrical activity. In recent years, advancements in neuroscience, signal processing, and machine learning have improved the accuracy, responsiveness, and usability of BCIs, opening the door to applications in medical rehabilitation, gaming, military, and human augmentation. Additionally, BCIs are being explored as therapeutic tools for treating various neurological diseases, for example, epilepsy, Parkinson's disease, and stroke-related disabilities.

### SUMMARY

Apparatus and associated methods relate to multi-faceted implantable physiological interface system including an array of panels each panel including a substrate defining opposing first and second sides, a bioprocessor supported on the first side and an electrode operatively coupled to the substrate on the second side. In an illustrative example, the substrate of each of the panels joins the substrate of at least one other adjacent panel such that the array of panels collectively define an outer enclosure having an internal volume configured to house one or more internal components. The internal components, may, for example, include sensors, actuators, therapeutic components, communication modules, data stores, security modules, thermal management units, and power sources. Various embodiments may advantageously provide multi-faceted, modular, adaptive physiological interfaces enabling precise monitoring, stimulation, and therapeutic interventions while dynamically conforming to a target environment.

Various embodiments may achieve one or more advantages. For example, some embodiments may advantageously increase the density of available channels to interface with physiological systems, enabling more detailed sensing and stimulation. Some embodiments may, for example, advantageously adjust to changes in brain architecture and neuroplasticity, ensuring long-term functionality and effectiveness. Some implementations may, for example, advantageously support multiple therapy modalities, including electrical, thermal, mechanical, and pharmaceutical delivery. Some embodiments may, for example, advantageously autonomously generate power. Some embodiments may, for example, advantageously monitor biomarkers, neural activity, and other physiological signals, enabling advanced diagnostics for diseases. Some implementations may, for example, advantageously be delivered via minimally invasive methods. Some embodiments may, for example, advantageously integrate with external devices.

The details of various embodiments are set forth in the accompanying drawings and the description below. Other features and advantages will be apparent from the description and drawings, and from the claims.

### BRIEF DESCRIPTION OF THE DRAWINGS

FIG. 1 depicts an exemplary multi-faceted implantable physiological interface system (MFIPIS) employed in an illustrative use-case scenario.

FIG. 2 illustrates a detailed schematic view of an example set of bioprocessor module panels (BPMP) within a bioprocessor module array (BPMA).

FIG. 3 depicts exemplary BPMAs operably coupled to a sheet.

FIG. 4 depicts an exemplary BPMA operably coupled to a shaft.

FIG. 5 depicts exemplary BPMAs operably coupled to a cable.

FIGS. 6A-6D illustrate example embodiments of various three-dimensional arrangements of a BPMA.

FIG. 7 illustrates an exemplary MFIPIS employed in an illustrative use-case scenario depicting a conceptual diagram of dynamically assessing the impedance of individual BPMPs within a MFIPIS to determine which BPMPs maintain optimal contact with a target area.

FIG. 8 is a block diagram depicting an exemplary BPMP and exemplary internal components within a BPMA's internal volume.

FIG. 9 is a flowchart illustrating an exemplary method of monitoring physiological activity and providing diagnostic feedback including exemplary operations that may be performed by a bioprocessor module (BPM).

FIG. 10 is a flowchart illustrating an exemplary method of activating one or more therapeutic components to deliver a therapeutic output including exemplary operations that may be performed by a BPM.

FIG. 11 is a flowchart illustrating an exemplary method of adapting an active panel configuration based on real-time contact conditions including exemplary operations that may be performed by a BPM.

FIG. 12 is a flowchart illustrating an exemplary method of continuously maintaining the temperature of a BPMA within a safe operating range including exemplary operations that may be performed by a BPM.

FIG. 13 is a flowchart illustrating an exemplary method of continuously monitoring and adapting power distribution and consumption of one or more power sources including exemplary operations that may be performed by a BPM.

Like reference symbols in the various drawings indicate like elements.

### DETAILED DESCRIPTION OF ILLUSTRATIVE EMBODIMENTS

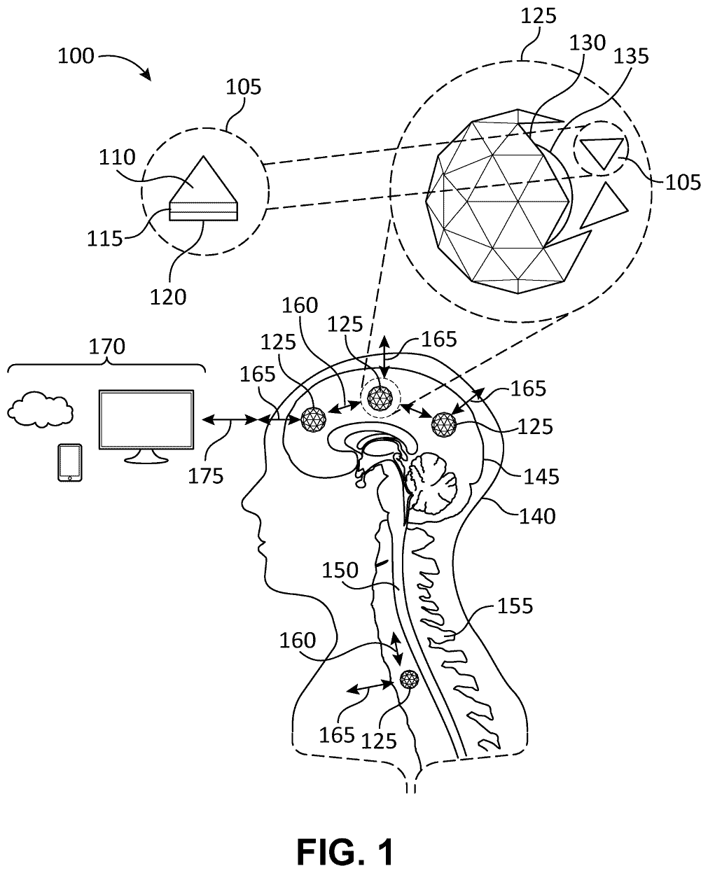

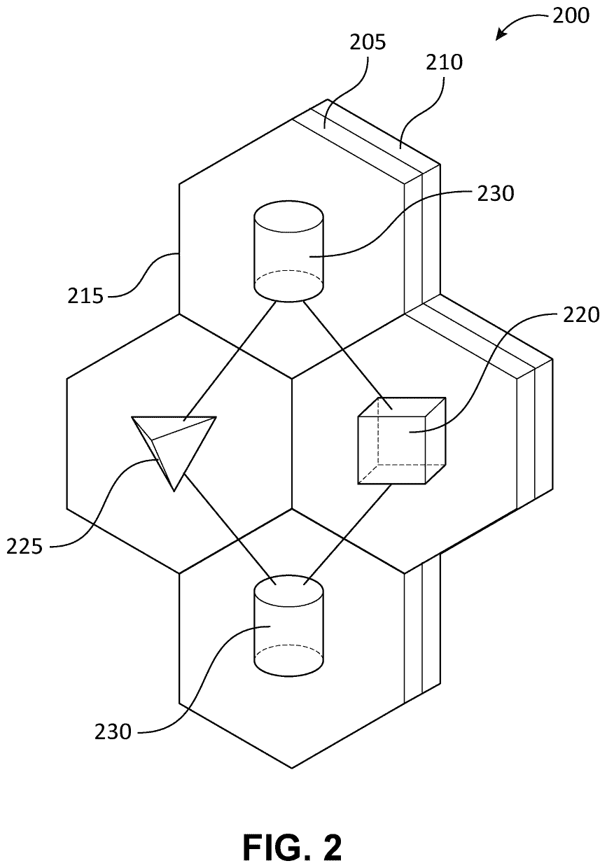

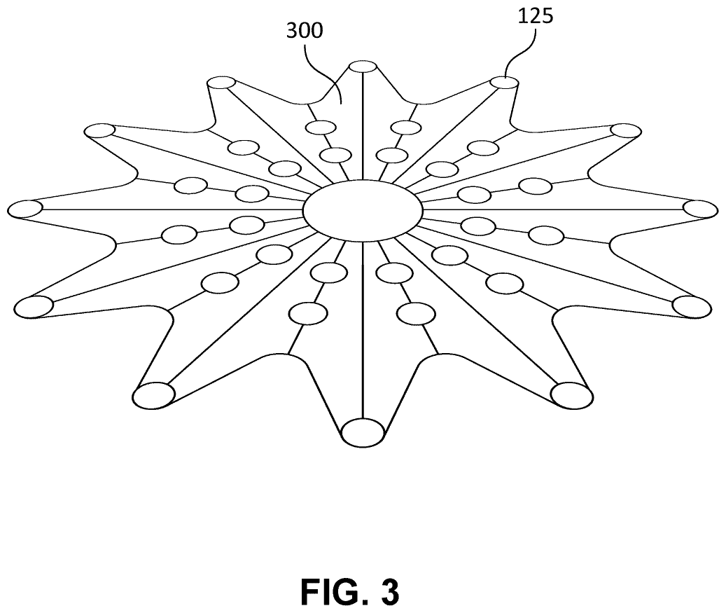

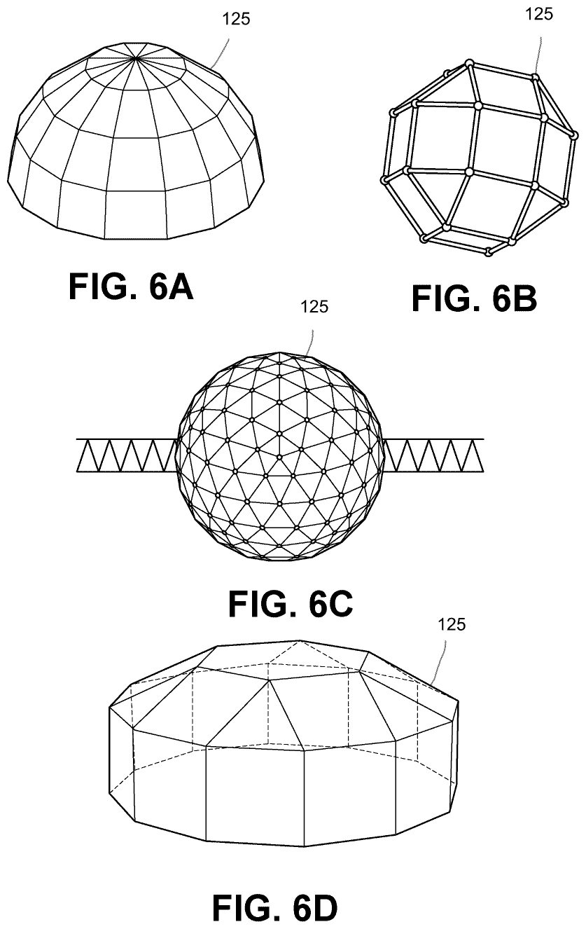

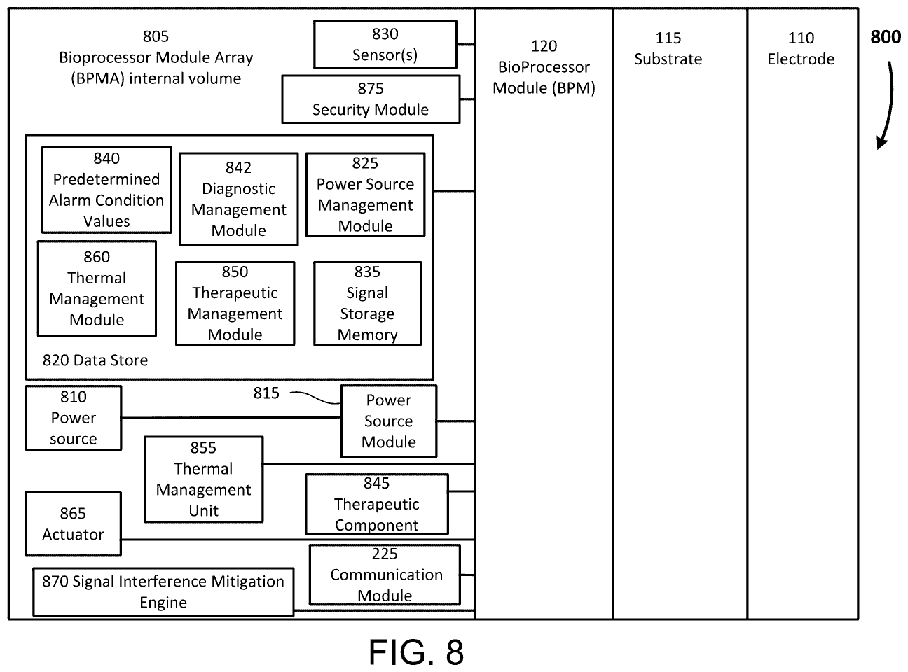

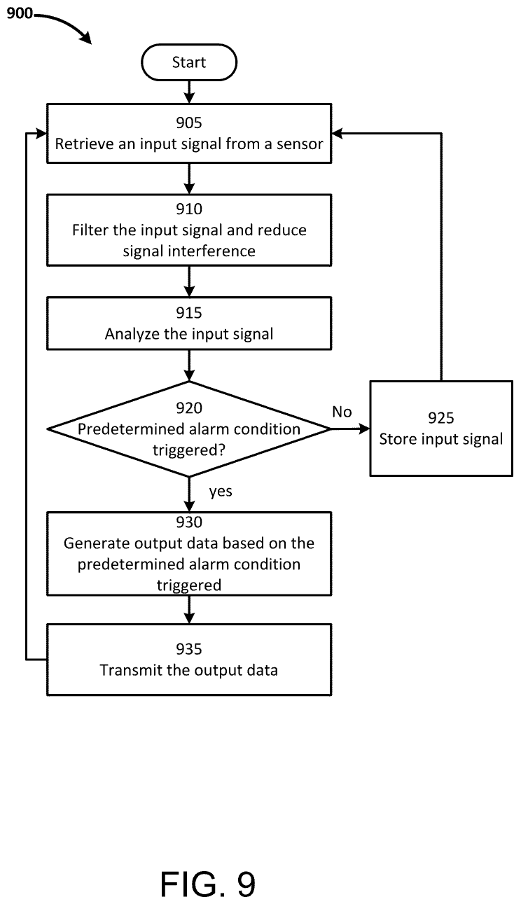

To aid understanding, this document is organized as follows. First, to help introduce discussion of various embodiments, multi-faceted implantable physiological interface system (MFIPIS) is introduced with reference to FIGS. 1-2. Second, that introduction leads into a description with reference to FIGS. 3-5 of some exemplary embodiments of delivery modules of bioprocessor module arrays (BPMA). Third, with reference to FIGS. 6A-6D, example embodiments of various three-dimensional arrangements of a BPMA are introduced. Fourth, with reference to FIG. 8, the discussion turns to an exemplary block diagram of a BPMP and exemplary internal components within a BPMA's internal volume. Fifth, and with reference to FIGS. 9-13, this document describes exemplary methods that may be performed by a bioprocessor module (BPM). Finally, the document discusses further embodiments, exemplary applications and aspects relating to a MFIPIS.

FIG. 1 depicts an exemplary multi-faceted implantable physiological interface system (MFIPIS) 100 employed in an illustrative use-case scenario. The MFIPIS 100 includes a bioprocessor module panel (BPMP) 105. The BPMP 105 includes an electrode 110 as the BPMP's 105 outer surface. The electrode 110 may, for example, advantageously facilitate the interaction between the BPMP 105 and a target area of the patient 140, enabling the BPMP 105 to sense electrical activity and deliver targeted stimulation.

The electrode 110 operably couples a substrate 115 such that the substrate is positioned on an inner surface of the BPMP 105. The substrate 115 may, for example, include silicone. The substrate 115 may, for example, advantageously enhance the structural integrity of the BPMP 105. The substrate 115 may, for example, advantageously contribute to the biocompatibility of the BPMP 105.

The substrate 115 operably couples a bioprocessor module (BPM) 120. The BPM 120 may, for example, include a microcontroller that executes instructions stored in a memory, a digital signal processor that performs mathematical operations on the signals, a field-programmable gate array that implements logic functions on the signals, and/or an artificial neural network that mimics the neural processing of the brain. The BPM 120 may, for example, advantageously enable the BPMP 105 to process signals and communicate with external systems or other BPMPs 105. The BPM 120 may, for example, advantageously provide an identification to the BPMP 105.

The BPMP 105 may, for example, join the substrate 115 of at least one other adjacent BPMP 105, such that when a plurality of BPMPs 105 join together, a bioprocessor module array (BPMA) 125 forms. The BPMPs 105 may, for example, join together via being bonded together using ultraviolet (UV) light to create a unified structure. The arrangement of BPMPs 105 in a BPMA 125 may, for example, be connected by inter-BPMP links. Linking may, for example, include through-silicon vias (TSVs), micro-bumps, and/or solder balls. Inter-BPMP links may, by way of example and not limitation, provide electrical, thermal, and/or mechanical coupling between the BPMPs 105. In some embodiments, a method for BPMA 125 assembly may include folding of BPMPs 105 (e.g., using origami techniques). For example, BPMPs 105 of a BPMA 125 may be connected via micro-hinges. Certain embodiments may be configured to secure BPMPs 105 in place once folded into the desired configuration. For example, adhesive layers may be configured to bond BPMPs 105 together at contact points. In some implementations, BPMPs 105 may, for example, be aggregated by an externally applied magnetic field and then cured using UV light, temperature, or other methods to form rigid structures that make up the BPMA 125. In some embodiments, BPMPs 105 may, for example, include alignment markers to ensure accurate positioning of BPMPs 105 relative to each other during assembly into the BPMA 125.

The substrate 115 may, for example, advantageously act as an insulator between the electrodes 110, ensuring electrical isolation and preventing signal interference or spread. Additionally, the substrate 115 may, for example, advantageously provide flexibility, enabling the BPMP 105 to adapt to various shapes and configurations during assembly of the BPMA 125. The BPM 120 may, for example, advantageously provide a unique identification to enable spatial recognition of the BPMP 105 such that the BPMP 105 may be tracked and recognized within the BPMA 125. Spatial recognition may, for example, advantageously enable the BPMP 105 to dynamically activate or deactivate based on sensing wants, ensuring precise functionality and efficient signal processing.

The BPMA 125 may, for example, define an outer enclosure having an internal volume 130 configured to house one or more internal components 135. As will be described in more detail with reference to FIGS. 2 and 8, internal components 135 may, for example, include sensors, actuators, therapeutic components, communication modules, data stores, security modules, thermal management units, and power sources. These components collectively may, for example, advantageously enable the BPMA 125 to perform complex functions such as sensing neural activity, stimulation, delivering drugs, transmitting data, and adapting to dynamic biological environments.

The use-case scenario depicted in FIG. 1 may, for example, include a medical scenario such that the MFIPIS 100 is disposed in a patient 140. For example, the MFIPIS 100 may integrate with biological tissues in advanced neurotechnology applications. The patient 140 may, for example, include a brain 145, spinal cord 150 and cerebrospinal fluid (CSF) space 155. The MFIPIS 100 may, for example, include a plurality BPMAs 125 disposed within a target region of the patient 140. For example, the target region may include a region of the brain 145 (e.g., brain ventricles). The target region may, for example, include a region of the spinal cord 150. The target region may, for example, include a region within the CSF space 155. The target region may, for example include a nerve.

In certain embodiments, BPMAs 125 may be placed in various locations. For example, BPMAs 125 may be disposed in and/or about the parenchyma. For example, BPMAs 125 may be disposed in and/or about the CSF. For example, BPMAs 125 may be disposed in and/or about vessels (e.g., blood vessels, lymphatic vessels). For example, BPMAs 125 may be disposed in and/or about soft tissue. For example, BPMAs 125 may be disposed in and/or about peripheral nerves. BPMAs 125 may, for example, be delivered endovascularly but deployed by puncturing the vessel wall and placing the spheres either with a subdural, subarachnoid space adjacent to or within the brain or along a Virchow Robbins space.

BPMAs 125 may, for example, include one or more communication modules (not depicted in FIG. 1). The one or more communication modules may, for example, be configured to communicably couple the BPMAs 125 within the MFIPIS 100 to each other, as is indicated by the arrows 160. The one or more communication modules may, for example, be configured to communicably couple the BPMAs 125 within the MFIPIS 100 to an external interface 170, as indicated by the arrows 165. The external interface 170 may, for example, be configured to communicably couple the MFIPIS 100, as indicated by the arrows 175, such that the BPMA 125 is configured to be remotely monitored via the external interface 170.

FIG. 2 illustrates a detailed schematic view of an example set of bioprocessor module panels 200 (BPMPs) within the BPMA 125. The set of BPMPs 200 includes individual BPMPs 205. Each of the BPMPs 205 may, for example, include a substrate, electrode, and a bioprocessor module (BPM) arranged in a substantially similar arrangement to the BPMP 105, electrode 110, substrate 115, and BPM 120. The BPMP 205 may, for example, include a coating 210 configured to protect the BPMP 205 from biological degradation and improve the BPMP's 205 biocompatibility. The coating 210 may, for example, advantageously improve conductivity of the BPMPs 205. The coating 210 may, for example, advantageously reduce the risk of infection that may be caused by the BPMPs 205. The coating may, for example, include a hydrogel.

The set of BPMPs 200 includes an internal volume 215 configured in a substantially similar arrangement to the internal volume 130. The internal volume 215 may, for example, be configured to house internal components (e.g., internal components 135). The internal components may, for example, include a power source 220. The power source 220 may, for example, be configured to power the BPMA 125. The power source 220 may, for example, include a battery that stores electrical energy. The power source 220 may, for example, include a capacitor that charges and discharges electrical energy. The power source 220 may, for example, include a solar cell that converts light into electrical energy. The power source 220 may, for example, include a wireless power receiver that receives electrical energy from an external source.

The internal components may, for example, include a communication module 225. The communication module 225 may, for example, be configured to communicably couple an external interface (e.g., external interface 170). The communication module 225 may, for example, be configured to communicably couple other BPMAs 125 within a MFIPIS 100. For example, the BPMAs 125 may be communicably coupled by one or more physical conductors. The BPMAs 125 may be communicably coupled wirelessly.

The communication module 225 may, for example, include a transmitter configured to send data. Data may, for example, be transmitted using electromagnetic waves. Data may, for example, be transmitted via an optical transmitter (e.g., that sends the data using photons). Data may, for example, be transmitted via a magnetic transmitter (e.g., that sends the data using magnetic fields). Data may, for example, be transmitted via a piezoelectric transmitter (e.g., that sends the data using mechanical vibrations).

Some embodiments include BPMPs 205 each with a communication module 225 configured to communicate with each other. For example, the BPMPs 205 may use a network-on-chip (NoC) protocol. The NoC protocol may enable high-bandwidth, low-latency, and scalable data transfer between the BPMPs 205. The NoC protocol may use packet switching, circuit switching, and/or hybrid switching techniques. The NoC protocol may support various topologies, such as mesh, torus, ring, tree, and/or hypercube.

Some embodiments of the communication module 225 may be configured to establish laser connectivity. For example, optical components may be configured to transmit data via laser beams, facilitating high-speed communication.

Some implementations of the communication module 225 may be configured to enable near-field communication from BPMAs 125 to peripheral units (e.g., adjacent to nerves). For example, wireless communication modules may be configured to pair BPMAs 125 with external interfaces for data exchange.

Some embodiments of the communication module 225 may be configured to enable multiplexing of signals for efficient communication. For example, a multiplexer may be configured to combine multiple signal streams into a single channel for transmission.

Some implementations of the communication module 225 may include a wireless transceiver configured to communicate with an external device (e.g., external interface 170) and perform bidirectional data transmission between the communication module 225 and the external device. Communication methods by the communication module 225 may, for example, include Bluetooth. Communication methods by the communication module 225 may, for example, include RF signals. Communication methods by the communication module 225 may, for example, include infrared signals.

Some implementations may involve BPMAs 125 configured to interact with phone-based applications. Such embodiments may, for example, advantageously permit user interaction and/or control over BPMA 125 functions. The communication module 225 may, for example, advantageously ensure efficient data exchange between BPMAs 125 and external systems, enabling advanced sensing, therapy delivery, and real-time control.

The internal components (e.g., internal components 135) may, for example, include components 230. As will be explained in more detail with reference to FIG. 8, the components 230 may, by way of example, and not limitation, include sensors, actuators, therapeutic components, data stores, security modules, thermal management units, and power source modules.

FIG. 3 depicts exemplary BPMAs 125 operably coupled to a sheet 300. The sheet 300 may, for example, operably couple multiple (e.g., hundreds, thousands) of BPMAs 125. The sheet 300 may, for example, advantageously enable the MFIPIS 100 to cover a large surface area, enabling the placement of multiple spheres for sensing and stimulation across a wide region of the target area. The sheet 300 may, for example, advantageously enable the MFIPIS 100 to be laid directly on a target area, simplifying the delivery process. The sheet 300 may, for example, advantageously enable the MFIPIS 100 to adapt to different anatomical locations, making them suitable for various medical applications.

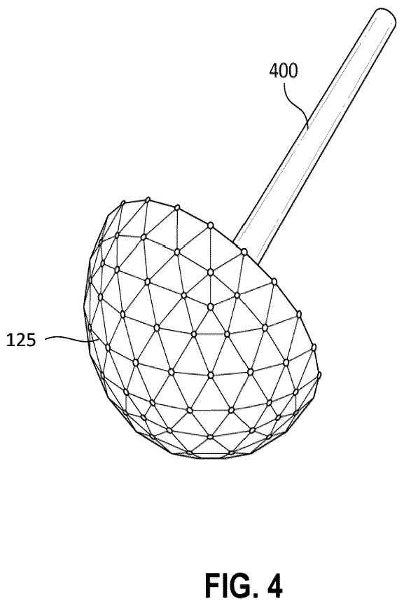

FIG. 4 depicts an exemplary BPMA 125 operably coupled to a shaft 400. The shaft 400 may, for example, advantageously enable precise insertion into specific target regions or depths, enabling focused sensing and stimulation. The shaft 400 may, for example, advantageously facilitate access to deeper neurological structures that may not be reachable with surface-based configurations. The shaft 400 may, for example, advantageously provide a compact and minimally invasive design, making it suitable for applications requiring smaller insertion points.

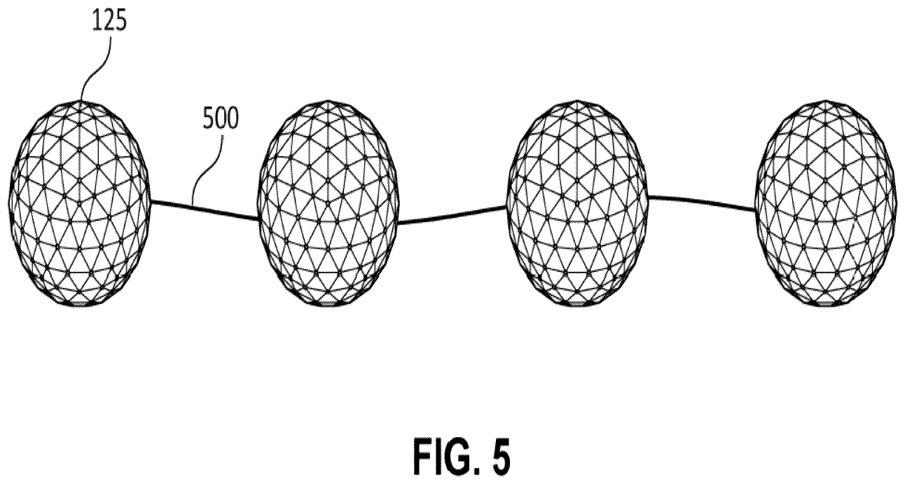

FIG. 5 depicts exemplary BPMAs 125 operably coupled to a cable 500. The cable 500 may, for example, include a string. The cable 500 may, for example, include a cord. The cable 500 may, for example, include a wire. The cable 500 may, for example, operably couple multiple (e.g., hundreds, thousands) of BPMAs 125. The cable 500 may, for example, advantageously provide a flexible structure, enabling BPMAs 125 to be positioned along curved or complex anatomical pathways, such as around peripheral nerves or the spinal cord. The cable 500 may, for example, advantageously thread through target regions, enabling precise placement of spheres in hard-to-reach areas. The cable 500 may, for example, advantageously enable insertion through small openings, reducing the invasiveness of the procedure and minimizing tissue damage.

FIGS. 6A-6D illustrate example embodiments of various three-dimensional arrangements of the BPMA 125. Each of the BPMPs 105 in the BPMA 125 may, for example, lie in a different plane. BPMPs 105 may, for example, be arranged in a buckyball-like structure, such as a geodesic dome composed of geometrical shapes, forming a substantially spherical shape. BPMPs 105 may, for example, be configured to form hexagonal or triangular shapes, which enable efficient packing and tessellation in a BPMA 125. The BPMA 125 may, for example, initially be connected in a planar arrangement before folding into 3D structures. BPMP's 105 may, for example, be delivered in string-like formations for flexible placement and potential reconfiguration post-deployment which may, advantageously enable the BPMA 125 to wrap around a target area, such as peripheral nerves or the spinal cord. The BPMA 125 may, for example, include hollow shapes, such as, for example, carbon nanotube-based configurations. A 3D structure of the BPMA 125 may, for example, include a stack of BPMPs 105 that are vertically aligned and connected by inter-BPMP links.

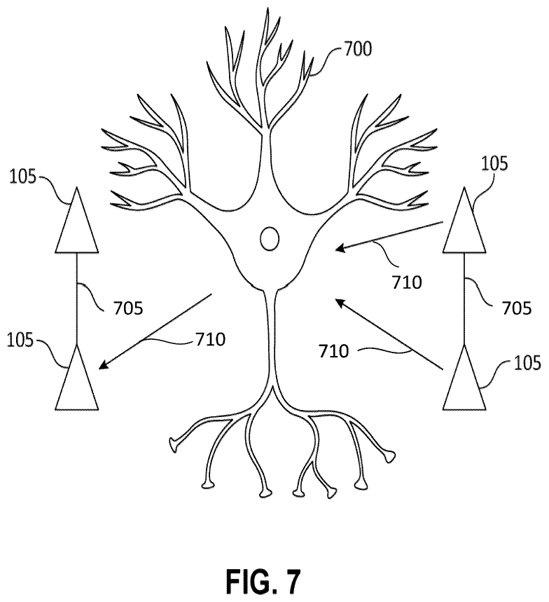

FIG. 7 illustrates the exemplary MFIPIS 100 employed in an illustrative use-case scenario depicting a conceptual diagram of dynamically assessing the impedance of individual BPMPs 105 within the MFIPIS 100 to determine which BPMPs 105 maintain contact with a target area 700. The target area may, for example, include a section of the nervous system. BPMPs 105 may, for example, be delivered to the target area by a delivery module 705. The delivery module 705 may, for example, include the sheet 300. The delivery module 705 may, for example, include the shaft 400. The delivery module 705 may, for example, include the cable 500.

Dynamically assessing the impedance of BPMPs 105 within the MFIPIS 100 may, for example, involve measuring of each BPMP's 105 contact with the surrounding target area, as is indicated by the arrows 710, in real time. This process may, for example, advantageously help identify which BPMPs 105 maintain contact with the target area, ensuring effective sensing and stimulation.

Dynamically assessing the impedance of BPMP's 105 within the MFIPIS 100 may, for example, involve continuously measuring the electrical resistance or impedance of each BPMP's 105 contact with the surrounding target area 700. Impedance serves as an indicator of the quality of the connection between the BPMPs 105 and the target area 700, with lower impedance typically signifying better contact and signal transmission. The MFIPIS 100 may, for example, use real-time feedback to analyze impedance data from all BPMPs 105, enabling the MFIPIS 100 to computationally identify which BPMPs 105 maintain optimal contact. BPMPs 105 with poor contact may, for example, be deactivated or reassigned, while those with better contact are selected for active use. This dynamic process may, for example, advantageously ensure that the MFIPIS 100 adapts to changes in tissue conditions, such as movement or shifts in brain architecture, by updating the selection of active BPMPs 105 in real time.

FIG. 8 is a block diagram depicting an exemplary BPMP 800 and exemplary internal components (e.g., internal components 135) within a BPMA's 125 internal volume 805. The BPMP 800 includes the electrode 110. The BPMP 800 includes the substrate 115. The BPMP 800 includes the BPM 120. The BPMP 800 may, for example, be configured in a substantially similar arrangement to the BPMP 105 and BPMP 205. The internal volume 805 may, for example, include the communication module 225.

The internal volume 805 may, for example, include a power source 810. The power source 810 may, for example, be configured in a substantially similar arrangement to the power source 220. The power source 810 may, for example, operably couple a power source module 815 configured to monitor the power level of the power source and charge the power source 810. The power source module 815 may, for example, operably couple the BPM 120. The BPM 120 may, for example, operably couple a data store 820. The data store 820 may, for example, include a power source management module 825 configured to provide instructions to operate the power source module 815. The BPM 120 may, for example, transmit instructions to operate the power source module 815 from the power source management module 825 to the power source module 815.

The power source module 815 may, for example, be configured to harvest power based on temperature gradients. For example, the power source module 815 may include a thermoelectric generator (TEG) coupled to one or more BPMPs 800. The TEG may, for example, convert heat into electrical energy. In some examples, the power source module 815 may charge the power source 810 by motion-activated power. In some implementations, the power source module 815 may charge the power source 810 by using CSF electrolytes (e.g., NaCl) to create a ‘bio-battery’. Some implementations of the power source module 815 may, for example, harvest energy from variations in body temperature (e.g., diurnal cycles).

The internal volume 805 may, for example, include one or more sensors 830 configured to monitor physiological signals. The one or more sensors 830 operably couple the BPM 120. The BPM 120 may, for example, transmit data collected from the one or more sensors 830 to the data store 820. The data store 820 may, for example, include a signal storage memory 835 configured to store data collected from the one or more sensors 830. The data store 820 may, for example, include predetermined alarm condition values 840. The predetermined alarm condition values 840 may, for example, include values of data that when measured by the one or more sensors 830, trigger an alarm condition. An alarm condition may, for example, represent a measurement from the one or more sensors 830 that indicates abnormal physiological behavior. The data store 820 may, for example, include a diagnostic management module 842 configured to store instructions to determine a potential medical diagnosis based on a triggered alarm condition.

The BPM 120 may, for example, transmit data collected from the one or more sensors 830 to the predetermined alarm condition values 840 to determine whether a predetermined alarm condition has been triggered. If a predetermined alarm condition has been triggered, the BPM 120 may, for example, transmit a signal representing the triggered predetermined alarm condition to the diagnostic management module 842 and/or to the communication module 225. The diagnostic management module 842 may, for example, determine a medical diagnosis or a potential medical diagnosis based on the triggered alarm condition. The communication module 225 may, for example, transmit the signal representing the triggered predetermined alarm condition or the potential medical diagnosis to an external device (e.g., external interface 170) or another BPMA 125 in the MFIPIS 100.

The one or more sensors 830 may include mechanical sensors (e.g., force, pressure, fluid flow, displacement). The one or more sensors 830 may, for example, include positioning sensors (e.g., proximity, location, orientation, velocity, acceleration). For example, the one or more sensors 830 may include inertial measurement units (IMUs). The one or more sensors 830 may include accelerometers. The one or more sensors 830 may, for example, include gyroscopes. The one or more sensors 830 may, for example, include thermal sensors. The one or more sensors 830 may, for example, include electromagnetic sensors. The one or more sensors 830 may, for example, include analyte sensors (e.g., lab-on-a-chip).

Some embodiments of the one or more sensors 830 include sensors configured to measure electrical activity of a brain. For example, some embodiments may include a sensor array comprising a plurality of sensors configured to detect electrical signals from a brain surface. For example, the sensors may be disposed on and/or about BPMPs (e.g., BPMP 105 and BPMP 205). The sensor array may be flexible and conformable to the shape of the brain surface. For example, the individual BPMAs 125 may move, thereby conforming the MFIPIS 100 to shape of the target surface while maintaining communication between the individual BPMAs 125.

The one or more sensors 830 may, for example, include a sensor configured to monitor a concentration and/or effect of the drugs or substances in the brain. The one or more sensors 830 may, for example, include a biosensor that detects the presence or amount of the drugs or substances, a pH sensor that measures the acidity or alkalinity of the brain tissue, a temperature sensor that measures the thermal changes in the brain, and/or a pressure sensor that measures mechanical forces.

The BPM 120 may, for example, operably couple a therapeutic component 845 configured to deliver a medical therapy to a target area of a patient. The data store 820 may, for example, include a therapeutic management module 850. The therapeutic management module 850 may, for example, be configured to store instructions to determine a medical therapy or a potential medical therapy based on a triggered alarm condition or a determined medical diagnosis. For example, the BPM 120 may transmit a signal representing a triggered alarm condition or a determined medical diagnosis to the therapeutic management module 850. Based on the signal representing a triggered alarm condition or a determined medical diagnosis, the therapeutic management module 850 may, for example, determine a medical therapy to treat the triggered alarm condition or a determined medical diagnosis. The BPM 120 may, for example, transmit a signal representing the determined medical therapy to the therapeutic component 845 such that the therapeutic component 845 activates to deliver a treatment. The therapeutic component 845 may, for example, deactivate and cease delivering the treatment once the triggered alarm condition is no longer triggered.

The therapeutic component 845 may, for example, include an electrode that delivers electrical currents or pulses to the neurons. The therapeutic component 845 may, for example, include a transducer that converts mechanical, optical, and/or acoustic signals into neural signals. The therapeutic component 845 may, for example, include a light source that emits light to activate or inhibit the neurons. In some implementations, the therapeutic component 845 may, for example, include thermal stimulus. In some implementations, the therapeutic component 845 may be configured to deliver gene therapy and/or synchronized pulses (e.g., electrical).

The therapeutic component 845 may, for example, be selectively operated to generate electromagnetic fields configured to disrupt and/or induce target brain activity. For example, some embodiments may be configured to treat epilepsy. Some embodiments may, for example, be deployed in the hippocampus.

Certain embodiments of the therapeutic component 845 may be configured to assist in the management of cerebrospinal fluid (CSF) flow. For example, the therapeutic component 845 may be configured as a valve system (e.g., networked) configured to regulate the pressure and/or circulation of CSF.

The therapeutic component 845 may, for example, include a drug delivery system that injects drugs or other substances to modulate neural activity. In some embodiments, the therapeutic component 845 may include a module configured to release drugs or other substances into the brain. The therapeutic component 845 may, for example, include a reservoir that stores the drugs or substances, a pump that controls the flow of the drugs or substances, a valve that regulates the opening and closing of the delivery module, and/or outlets that direct the drugs or substances to a target location.

The internal volume 805 may, for example, include a thermal management unit (TMU) 855 configured to monitor the temperature of the BPMA 125 or individual BPMPs 800 and maintain the temperature at a safe operating range. The data store 820 may, for example, include a thermal management module 860 configured to store instructions to maintain the temperature of the BPMA 125 or individual BPMPs 800 at a safe operating range. For example, the BPM 120 may transmit a signal representing temperature data of the temperature BPMA 125 or individual BPMPs 800 from the TMU 855 to the thermal management module 860. The thermal management module 860 may, for example, compare the data of the temperature BPMA 125 or individual BPMPs 800 to predetermined thermal thresholds corresponding to a safe operating range to determine whether the temperature data falls outside the predetermined thermal thresholds. Based on the determination that the temperature exceeds an upper threshold, the BPM 120 may, for example, send a signal to activate the TMU 855 to dissipate heat from the BPMA 125 or individual BPMPs 800 to return the temperature to a predetermined safe operating range.

The TMU 855 may, for example, include a heat sink configured to dissipate heat. The TMU 855 may, for example, include a thermal interface material configured to conduct heat away from hot spots within the BPMA 125 or individual BPMPs 800.

The internal volume 805 may, for example, include one or more actuators 865. The one or more actuators 865 may, for example, include electromagnetic components (e.g., voltage generator, current generator, inductor). For example, the one or more actuators 865 may, for example, be configured to generate electromagnetic signals (e.g., electric fields, magnetic fields, light).

The one or more actuators 865 may, for example, include mechanical actuators. (e.g., pneumatic driven, hydraulic driven, electronic driven, magnetic driven). For example, mechanical actuators may generate rotation and/or translation (e.g., of a valve, of a position of a BPMP 105 and/or BPMA 125). Mechanical actuators may, for example, dispense substances (e.g., pharmaceuticals). Mechanical actuators may, for example, include shape memory materials (e.g., polymers, alloys). The one or more actuators 865 may, for example, include fixation devices (e.g., selectively actuated protrusion(s) configured to engage tissue).

The internal volume 805 may, for example, include a signal interference mitigation engine 870. Some embodiments may be configured to reduce signal interference via the signal interference mitigation engine 870. For example, the signal interference mitigation engine 870 may be configured to preserve signal integrity across shared communication channels during multiplexing. The signal interference mitigation engine 870 may, for example, be configured to reduce noise of signals or reduce overlapping signals.

The internal volume 805 may, for example, include a security module 875 configured to protect the data or instructions from unauthorized access or modification. The security module 875 may, for example, include a cryptographic processor that encrypts or decrypts the data or instructions using a secret key. The security module 875 may, for example, include a biometric sensor that authenticates the identity of a user based on a physical or behavioral trait. The security module 875 may, for example, include a firewall that blocks unwanted network traffic. The security module 875 may, for example, include a tamper-resistant circuit that disables the BPMA 125 if it detects an intrusion attempt.

FIG. 9 is a flowchart illustrating an exemplary method 900 of monitoring physiological activity and providing diagnostic feedback including exemplary operations that may be performed by the BPM 120. In a step 905, the BPM 120 retrieves an input signal from the one or more sensors 830. In a step 910, the BPM 120 transmits the input signal to filter and reduce signal interference.

In a step 915, the BPM 120 transmits the input signal to the data store 820 to be analyzed. In a step 920, the analysis of the input signal leads to a decision point, where based on the analysis it is decided whether a predetermined alarm condition has been triggered. For example, an alarm condition may, for example, represent a measurement from the one or more sensors 830 that indicates abnormal physiological behavior. If a predetermined alarm condition is determined to not have been triggered, the method 900 proceeds to a step 925. In a step 925, the BPM 120 stores the input signal in the data store 820. After step 925, the method 900 reverts to step 905.

If a predetermined alarm condition is determined to have been triggered, the method 900 proceeds to a step 930. In a step 930, the BPM 120 generates output data based on the predetermined alarm condition. For example, the output data may, for example, include a signal representing the triggered predetermined alarm condition or a medical diagnosis or a potential medical diagnosis based on the triggered alarm condition.

In a step 935, the BPM 120 transmits the output data. For example, the BPM 120 may, for example, transmit the output data to the communication module 225. The communication module 225 may, for example, transmit the signal representing the triggered predetermined alarm condition or the potential medical diagnosis to an external device (e.g., external interface 170) or another BPMA 125 in the MFIPIS 100. After step 935, the method 900 reverts to step 905.

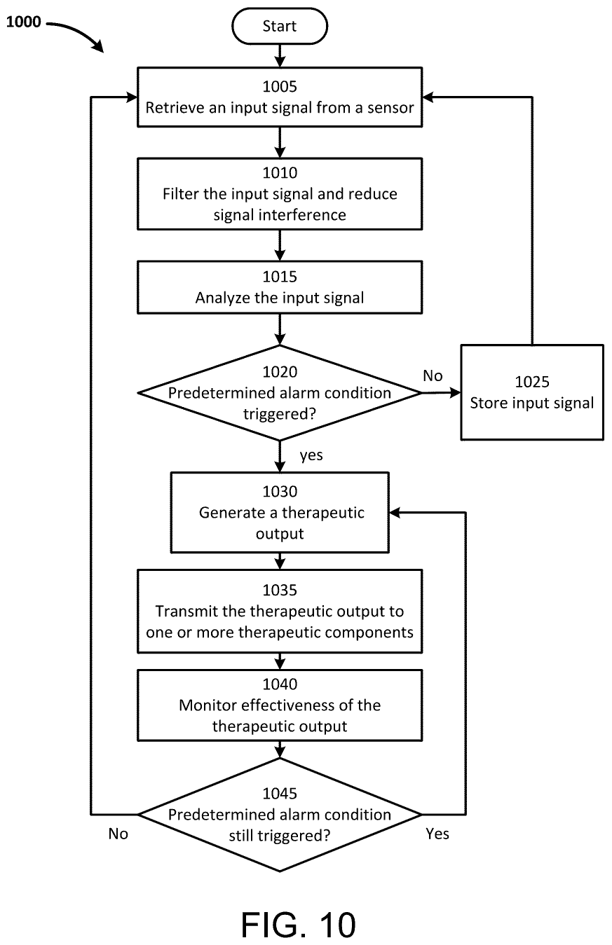

FIG. 10 is a flowchart illustrating an exemplary method 1000 of activating one or more therapeutic components 845 to deliver a therapeutic output including exemplary operations that may be performed by the BPM 120. In a step 1005, the BPM 120 retrieves an input signal from the one or more sensors 830. In a step 1010, the BPM 120 transmits the input signal to filter and reduce signal interference.

In a step 1015, the BPM 120 transmits the input signal to the data store 820 to be analyzed. In a step 1020, the analysis of the input signal leads to a decision point, where based on the analysis it is decided whether a predetermined alarm condition has been triggered. For example, an alarm condition may, for example, represent a measurement from the one or more sensors 830 that indicates abnormal physiological behavior. If a predetermined alarm condition is determined to not have been triggered, the method 1000 proceeds to a step 1025. In a step 1025, the BPM 120 stores the input signal in the data store 820. After step 1025, the method 1000 reverts to step 1005.

If a predetermined alarm condition is determined to have been triggered, the method 1000 proceeds to a step 1030. In a step 1030, the BPM 120 generates a signal representing a therapeutic output data based on the predetermined alarm condition. For example, the therapeutic output may, for example, include a signal representing a medical therapy to treat the predetermined alarm condition.

In a step 1035, the BPM 120 transmits the therapeutic output to one or more therapeutic components 845 such that the therapeutic component 845 activates to deliver a treatment to treat the predetermined alarm condition. In a step 1040, one or more sensors 830 monitor the effectiveness of the therapeutic output in treating the predetermined alarm condition such that the predetermined alarm condition is no longer triggered.

In a step 1045, a decision point is reached where it is determined whether the predetermined alarm condition is still triggered. If the predetermined alarm condition is determined to still be triggered, then the method 1000 reverts to a step 1030. If the predetermined alarm condition is determined to no longer be triggered, then the method 1000 reverts to step 1005.

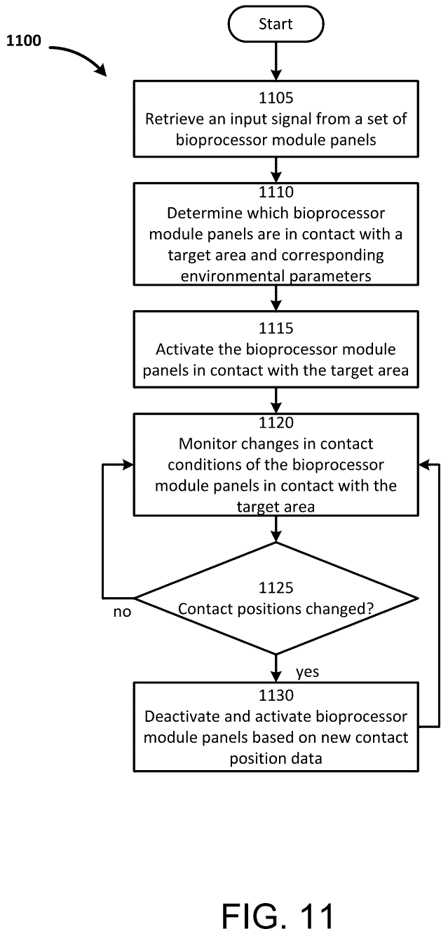

FIG. 11 is a flowchart illustrating an exemplary method 1100 of adapting an active panel configuration based on real-time contact conditions including exemplary operations that may be performed by the BPM 120. The active panel configuration may, for example, include a set of BPMPs 105 that are activated to function and a set of BPMPs 105 that are deactivated.

In a step 1105, the BPM 120 retrieves an input signal from a set of BPMPs 105, the signal representing that the set of BPMPs 105 are in contact with a target area. In a step 1110, the BPM 120 determines which BPMPs 105 are in contact with the target area and determines corresponding environmental parameters. Corresponding environmental parameters may, for example, include temperature of the target area, mechanical forces such as fluid pressor of the target area, the presence or concentration of chemicals or substances in the target area, neural activity (e.g., electrical signals) in the target area, and/or pH levels of the target area. These environmental parameters may, for example, advantageously, help the MFIPIS 100 dynamically adapt its functions, such as activating sensors, delivering therapy, or adjusting operations based on real-time feedback from the target area.

In a step 1115, the BPM 120 activates the BPMPs 105 in contact with the target area. Activating a BPMP 105 may, for example, refer to enabling or initiating its functionality to perform specific tasks or operations.

In a step 1120, the BPM 120 monitors changes in contact conditions of the BPMPs 105 in contact with the target area in real-time. Contact conditions may, for example, refer to the chemical, or environmental characteristics of the interaction between the BPMPs 105 and the target area. Monitoring changes in contact conditions may, for example, include monitoring changes in position and orientation of the BPMPs 105, environmental parameters, and/or interactions between the BPMPs 105 and the target area.

In a step 1125, a decision point is reached where the BPM 120 decides whether the contact position between the BPMPs 105 and the target area has changed. If the BPM 120 decides no, the contact position between the BPMPs 105 and the target area has not changed, the method 1100 reverts to step 1120. If the BPM 120 decides yes, the contact position between the BPMPs 105 and the target area has changed, the method 1100 proceeds to step 1130. In a step 1130, the BPM 120 deactivates and activates BPMPs 105 based on the new contact position data. For example, the BPM 120 may, for example, activate BPMPs 105 in contact with the target area, and deactivate BPMPs 105 no longer in contact with the target area. After step 1130, the method 1100 reverts to step 1120.

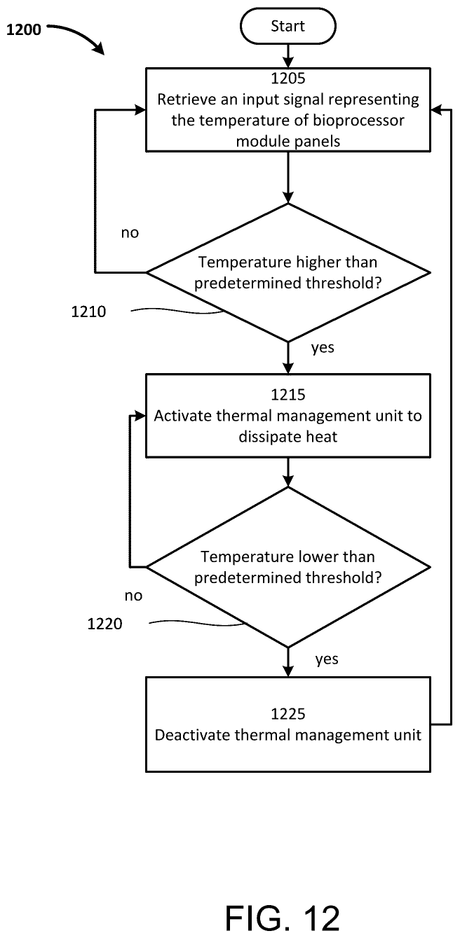

FIG. 12 is a flowchart illustrating an exemplary method 1200 of continuously maintaining the temperature of the BPMA 125 within a safe operating range including exemplary operations that may be performed by the BPM 120. In a step 1205, the BPM 120 retrieves an input signal representing the temperature of the BPMPs 105. The BPM 120 may, for example, retrieve the input signal from the TMU 855 or the one or more sensors 830.

In a step 1210, a decision point is reached where the BPM 120 determines whether the temperature of the BPMPs 105 is higher than a predetermined threshold, the predetermined threshold corresponding to a safe operating range. If the BPM 120 decides no, the temperature of the BPMPs 105 is not higher than a predetermined threshold, then the method 1200 reverts to step 1205. If the BPM 120 decides yes, the temperature of the BPMPs 105 is higher than a predetermined threshold, then the method 1200 proceeds to a step 1215. In step 1215, the BPM 120 activates the TMU 855 to dissipate the heat of the BPMPs 105.

In a step 1220, a decision point is reached where the BPM 120 determines whether the temperature of the BPMPs 105 is higher than a predetermined threshold, the predetermined threshold corresponding to a safe operating range. If the BPM 120 decides no, the temperature of the BPMPs 105 is not higher than a predetermined threshold, then the method 1200 reverts to step 1215. If the BPM 120 decides yes, the temperature of the BPMPs 105 is higher than a predetermined threshold, then the method 1200 proceeds to a step 1225. In step 1225, the BPM 120 deactivates the TMU 855. After step 1225, the method 1200 reverts to step 1205.

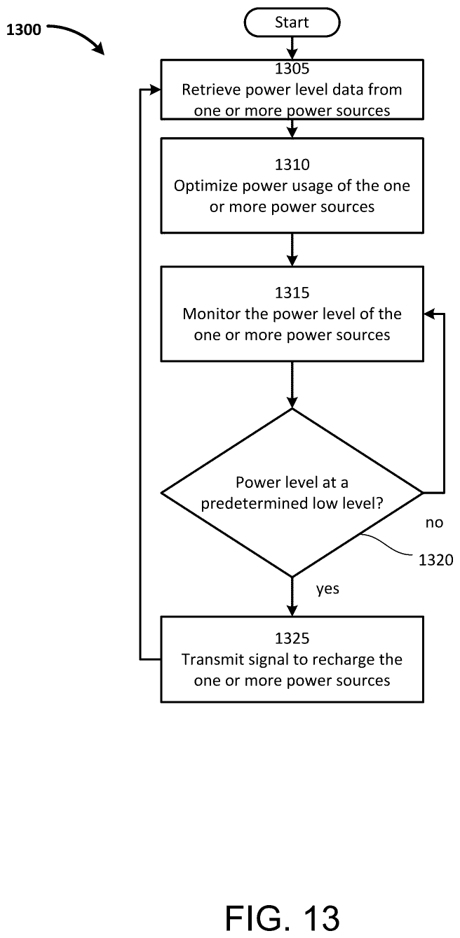

FIG. 13 is a flowchart illustrating an exemplary method 1300 of continuously monitoring and adapting power distribution and consumption of one or more power sources (e.g. power source 220 and power source 810) including exemplary operations that may be performed by the BPM 120. In a step 1305, the BPM 120 retrieves power level data from the one or more power sources. In a step 1310, the BPM 120, optimizes the power usage of the one or more power sources. Optimizing power usage may, for example include selective activation of BPMPs 105 and internal components 135 based on operational wants. Optimizing power usage may, for example include adaptive frequency and voltage scaling. Optimizing power usage may, for example include low-power states for idle internal components 135.

In a step 1315, the BPM 120 monitors the power level of the one or more power sources via the power source module 815. In a step 1320, the method 1300 reaches a decision point where the BPM 120 decides whether the power level of the one or more power sources is at a predetermined low level. If the BPM 120 decides no, the power level of the one or more power sources is not at a predetermined low level, then the method 1300 reverts to step 1315. If the BPM 120 decides yes, the power level of the one or more power sources is at a predetermined low level, then the method 1300 proceeds to step 1325. In a step 1325, the BPM 120 transmits a signal to the power source module 815 to recharge the one or more power sources. After step 1325, the method 1300 reverts to step 1305.

In an exemplary embodiment, BPMAs 125 may be configured as autonomous (e.g., self-powered, remotely coupled to a master system and/or other BPMAs 125). Autonomous BPMAs 125 may, for example, be released into the CSF. Autonomous BPMAs 125 may, for example, be selectively activated (e.g., if they are in a desired location).

In some implementations, BPMAs 125 may, for example, be disposed in and/or about optical structures (e.g., the eye, the optic nerve). For example, BPMAs 125 may be configured to communicate via the optic nerve. In some implementations, for example, BPMAs 125 may be embedded in contact lenses. External BPMAs 125 (such as in contact lens) may, by way of example and not limitation, be in operable communication with implanted BPMAs 125. For example, inter-BPMA 125 communication may be via physiological channels (e.g., nervous system, CSF). For example, inter-BPMA 125 communication may be via wireless signals. For example, inter-BPMA 125 communication may be wired. For example, inter-BPMA 125 communication may be via an external controller(s).

Some embodiments include a method of implanting the BPMAs 125. The method may, for example, include inserting a needle into the skull, advancing the needle through the brain tissue, deploying the BPMAs from the needle into the brain tissue, and retracting the needle from the skull. BPMAs 125 may, for example, be delivered via an injection with a delivery module configured to hold the BPMAs 125. Some embodiments of BPMAs 125 may, for example, be delivered to a target location in delivery modules. The delivery module may, for example, include the sheet 300. The delivery module may, for example, include the shaft 400. The delivery module may, for example, include the cable 500.

In some implementations, BPMAs 125 may be configured to dynamically adjust bandwidth allocation based on BPMPs 105 performance. For example, a performance monitoring system may be configured to optimize bandwidth usage in real-time.

BPMAs 125 may, for example, be adaptive to dynamic changes in the brain architecture and neuroplasticity changes that occur. The MFIPIS 100 may, for example, contribute to recursive stimulation paradigms for neuroplasticity, memory and cognition.

Some embodiments of the MFIPIS 100 may, for example, provide edge computing. For example, BPMAs 125 may provide edge computing. Edge computing at an BPMA 125 may advantageously increase response speed, increase computing power, and/or increase data autonomy. BPMAs 125 may be connected to a central and/or remote controller(s) via open and/or closed loop feedback.

The BPMAs 125 may, for example, be delivered as an embolization material (e.g., such as to fill the middle meningeal artery which overlies the motor cortex). BPMAs 125 may, for example, be delivered endovascularly on a shape memory alloy (coil/stent/otherwise).

BPMAs 125 may, for example, be adaptive to dynamic changes in the brain architecture and neuroplasticity changes that occur. The MFIPIS 100 may, for example, contribute to recursive stimulation paradigms for neuroplasticity, memory and cognition.

In some embodiments, the MFIPIS 100 may, for example, be configured to generate a phased array configured to steer current in one direction or another between two leads. In some implementations, the MFIPIS 100 may, for example, leverage AI and advanced algorithms, to advantageously enable the system to function adaptively and effectively in real-time, maintaining high performance in sensing and other tasks.

Although various embodiments have been described with reference to the figures, other embodiments are possible.

Although an exemplary system has been described with reference to FIG. 1, other implementations may be deployed in other industrial, scientific, medical, commercial, and/or residential applications.

The MFIPIS 100 may, for example be deployed in the medical industry, particularly for brain-computer interfaces, neural implants, and therapeutic devices. The MFIPIS 100 may, for example, be employed for monitoring and modulating neural activity, delivering targeted drug therapies, treating neurodegenerative diseases, and managing cerebrospinal fluid flow. Applications may, for example, include epilepsy treatment, neuroplasticity enhancement, and addressing conditions like cerebral palsy or spinal cord injuries. The MFIPIS 100 may, for example, be employed in personalized medicine and advanced diagnostics.

In scientific research, the MFIPIS 100 may, for example, be used to study brain activity, neuroplasticity, and cognitive processes. The MFIPIS 100 may, for example, provide real-time data on neural signals, enabling researchers to explore brain function and develop new insights into neurological disorders. The MFIPIS 100 may, for example, enable experimentation with various configurations, making it a potential tool in neuroscience, bioengineering, and computational modeling of brain activity.

The MFIPIS 100 may, for example be used in robotics for advanced sensory feedback and control systems, or in manufacturing processes where adaptive systems are used to respond to environmental changes. Its edge computing capabilities and modular design could also support automation and optimization in complex industrial systems.

In the commercial sector, the MFIPIS 100 could be employed in wearable technology, smart devices, and augmented reality systems. The MFIPIS 100 may, for example, be employed in health monitoring, cognitive enhancement, or immersive experiences. For example, the MFIPIS 100 could be integrated into smart glasses or contact lenses for augmented reality applications.

For residential use, the MFIPIS 100 could, for example, be integrated into smart home devices for health monitoring, environmental sensing, or personalized therapy. The MFIPIS 100 may, for example, be employed in applications like sleep tracking, stress management, or home-based rehabilitation.

In various embodiments, some bypass circuits implementations may be controlled in response to signals from analog or digital components, which may be discrete, integrated, or a combination of each. Some embodiments may include programmed, programmable devices, or some combination thereof (e.g., PLAs, PLDs, ASICs, microcontroller, microprocessor), and may include one or more data stores (e.g., cell, register, block, page) that provide single or multi-level digital data storage capability, and which may be volatile, non-volatile, or some combination thereof. Some control functions may be implemented in hardware, software, firmware, or a combination of any of them.

Computer program products may contain a set of instructions that, when executed by a processor device, cause the processor to perform prescribed functions. These functions may be performed in conjunction with controlled devices in operable communication with the processor. Computer program products, which may include software, may be stored in a data store tangibly embedded on a storage medium, such as an electronic, magnetic, or rotating storage device, and may be fixed or removable (e.g., hard disk, floppy disk, thumb drive, CD, DVD).

Although an example of a system, which may be portable, has been described with reference to the above figures, other implementations may be deployed in other processing applications, such as desktop and networked environments.

Temporary auxiliary energy inputs may be received, for example, from chargeable or single use batteries, which may enable use in portable or remote applications. Some embodiments may operate with other DC voltage sources, such as a 9V (nominal) batteries, for example. Alternating current (AC) inputs, which may be provided, for example from a 50/60 Hz power port, or from a portable electric generator, may be received via a rectifier and appropriate scaling. Provision for AC (e.g., sine wave, square wave, triangular wave) inputs may include a line frequency transformer to provide voltage step-up, voltage step-down, and/or isolation.

Although particular features of an architecture have been described, other features may be incorporated to improve performance. For example, caching (e.g., L1, L2, . . . ) techniques may be used. Random access memory may be included, for example, to provide scratch pad memory and or to load executable code or parameter information stored for use during runtime operations. Other hardware and software may be provided to perform operations, such as network or other communications using one or more protocols, wireless (e.g., infrared) communications, stored operational energy and power supplies (e.g., batteries), switching and/or linear power supply circuits, software maintenance (e.g., self-test, upgrades), and the like. One or more communication interfaces may be provided in support of data storage and related operations.

Some systems may be implemented as a computer system that can be used with various implementations. For example, various implementations may include digital circuitry, analog circuitry, computer hardware, firmware, software, or combinations thereof. Apparatus can be implemented in a computer program product tangibly embodied in an information carrier, e.g., in a machine-readable storage device, for execution by a programmable processor; and methods can be performed by a programmable processor executing a program of instructions to perform functions of various embodiments by operating on input data and generating an output. Various embodiments can be implemented advantageously in one or more computer programs that are executable on a programmable system including at least one programmable processor coupled to receive data and instructions from, and to transmit data and instructions to, a data storage system, at least one input device, and/or at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, which may include a single processor or one of multiple processors of any kind of computer. Generally, a processor will receive instructions and data from a read-only memory or a random-access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including, by way of example, semiconductor memory devices, such as EPROM, EEPROM, and flash memory devices; magnetic disks, such as internal hard disks and removable disks; magneto-optical disks; and CD-ROM and DVD-ROM disks. The processor and the memory can be supplemented by, or incorporated in, ASICs (application-specific integrated circuits).

In some implementations, each system may be programmed with the same or similar information and/or initialized with substantially identical information stored in volatile and/or non-volatile memory. For example, one data interface may be configured to perform auto configuration, auto download, and/or auto update functions when coupled to an appropriate host device, such as a desktop computer or a server.

In some implementations, one or more user-interface features may be custom configured to perform specific functions. Various embodiments may be implemented in a computer system that includes a graphical user interface and/or an Internet browser. To provide for interaction with a user, some implementations may be implemented on a computer having a display device. The display device may, for example, include an LED (light-emitting diode) display. In some implementations, a display device may, for example, include a CRT (cathode ray tube). In some implementations, a display device may include, for example, an LCD (liquid crystal display). A display device (e.g., monitor) may, for example, be used for displaying information to the user. Some implementations may, for example, include a keyboard and/or pointing device (e.g., mouse, trackpad, trackball, joystick), such as by which the user can provide input to the computer.

In various implementations, the system may communicate using suitable communication methods, equipment, and techniques. For example, the system may communicate with compatible devices (e.g., devices capable of transferring data to and/or from the system) using point-to-point communication in which a message is transported directly from the source to the receiver over a dedicated physical link (e.g., fiber optic link, point-to-point wiring, daisy-chain). The components of the system may exchange information by any form or medium of analog or digital data communication, including packet-based messages on a communication network. Examples of communication networks include, e.g., a LAN (local area network), a WAN (wide area network), MAN (metropolitan area network), wireless and/or optical networks, the computers and networks forming the Internet, or some combination thereof. Other implementations may transport messages by broadcasting to all or substantially all devices that are coupled together by a communication network, for example, by using omni-directional radio frequency (RF) signals. Still other implementations may transport messages characterized by high directivity, such as RF signals transmitted using directional (i.e., narrow beam) antennas or infrared signals that may optionally be used with focusing optics. Still other implementations are possible using appropriate interfaces and protocols such as, by way of example and not intended to be limiting, USB 2.0, Firewire, ATA/IDE, RS-232, RS-422, RS-485, 802.11 a/b/g, Wi-Fi, Ethernet, IrDA, FDDI (fiber distributed data interface), token-ring networks, multiplexing techniques based on frequency, time, or code division, or some combination thereof. Some implementations may optionally incorporate features such as error checking and correction (ECC) for data integrity, or security measures, such as encryption (e.g., WEP) and password protection.

In various embodiments, the computer system may include Internet of Things (IoT) devices. IoT devices may include objects embedded with electronics, software, sensors, actuators, and network connectivity which enable these objects to collect and exchange data. IoT devices may be in-use with wired or wireless devices by sending data through an interface to another device. IoT devices may collect useful data and then autonomously flow the data between other devices.

Various examples of modules may be implemented using circuitry, including various electronic hardware. By way of example and not limitation, the hardware may include transistors, resistors, capacitors, switches, integrated circuits, other modules, or some combination thereof. In various examples, the modules may include analog logic, digital logic, discrete components, traces and/or memory circuits fabricated on a silicon substrate including various integrated circuits (e.g., FPGAs, ASICs), or some combination thereof. In some embodiments, the module(s) may involve execution of preprogrammed instructions, software executed by a processor, or some combination thereof. For example, various modules may involve both hardware and software.

A number of implementations have been described. Nevertheless, it will be understood that various modifications may be made. For example, advantageous results may be achieved if the steps of the disclosed techniques were performed in a different sequence, or if components of the disclosed systems were combined in a different manner, or if the components were supplemented with other components. Accordingly, other implementations are contemplated within the scope of the following claims.

What is claimed is:

1. An implantable physiological interface device comprising:
an array of panels, each panel comprising:
a substrate defining opposing first and second sides;
a bioprocessor supported on the first side; and,
an electrode disposed on the second side operatively coupled to the bioprocessor; and,
wherein the substrate of each of the panels joins the substrate of at least one other adjacent panel, such that the array of panels collectively define an outer enclosure having an internal volume configured to house one or more internal components; and,
a data store comprising a program of instructions and operably coupled to the bioprocessor, such that, when the bioprocessor executes the program of instructions, the bioprocessor causes operations to be performed to interface with a physiological environment; and,
a delivery module configured to deliver the array of panels to a target area of a patient.

2. The implantable physiological interface device of claim 1, wherein each of the panels in the array lies in a different plane.

3. The implantable physiological interface device of claim 1, further comprising one or more internal components housed within the internal volume of the outer enclosure.

4. The implantable physiological interface device of claim 3, wherein the one or more internal components comprises:
a communication module communicably coupled to the bioprocessor and communicably coupled to an external interface such that the physiological interface device is configured to be remotely monitored via the external interface;
one or more sensors operably coupled to the bioprocessor; and,
wherein, the operations further comprise:
retrieve an input signal from the one or more sensors;
store the input signal in the data store;
based on the input signal, determine whether a predetermined alarm condition has been triggered;
generate output data based on the predetermined alarm condition triggered; and,
transmit the output data to the external interface,
such that the one or more sensors monitor physiological activity and provide diagnostic feedback.

5. The implantable physiological interface device of claim 3, wherein the one or more internal components comprise:
one or more sensors operably coupled to the bioprocessor;
one or more therapeutic components operably coupled to the bioprocessor; and,
wherein, the operations further comprise:
retrieve an input signal from the one or more sensors;
store the input signal in the data store;
based on the input signal, determine whether a predetermined alarm condition has been triggered;
generate a signal representing a therapeutic output based on the predetermined alarm condition triggered;
transmit the signal representing the therapeutic output to the one or more therapeutic components until the predetermined alarm condition is no longer triggered,
wherein receiving the signal representing the therapeutic output activates the one or more therapeutic components to deliver the therapeutic output.

6. The implantable physiological interface device of claim 1, wherein the operations further comprise:
retrieve an input signal from a set of panels that identifies which panels are in contact with a target area of a patient;
analyze the input signal and determine which panels are in contact with the target area and determine corresponding environmental parameters of the panels in contact with the target area;
based on the analyzed input signal, activate the set of panels in contact with the target area;
monitor changes in contact conditions between the set of panels in contact with the target area and the target area over a predetermined period;
recalibrate the set of panels by deactivating and activating panels in response to monitored changes in contact conditions;
wherein the operations are performed iteratively to continuously adapt an active panel configuration based on real-time contact conditions.

7. The implantable physiological interface device of claim 3, wherein the one or more internal components comprise:
one or more sensors operably coupled to the bioprocessor;
one or more thermal management units operably coupled to the bioprocessor; and,
wherein, the operations further comprise:
retrieve an input signal representing temperature data of the array of panels from the one or more sensors;
analyze the temperature data by comparing the input signal representing temperature data to one or more predetermined thermal thresholds corresponding to a safe operating range;
determine whether the temperature data falls outside the predetermined thermal thresholds;
in response to a determination that the temperature exceeds an upper threshold, activate the one or more thermal management units configured to dissipate heat from the array of panels;
determine whether the temperature returns to within the safe operating range;
wherein the operations are performed iteratively to continuously maintain the temperature within the safe operating range.

8. The implantable physiological interface device of claim 3, wherein the one or more internal components comprise:
a power source module operably coupled to the bioprocessor and configured to monitor and charge one or more power sources; and,
wherein, the operations further comprise:
retrieve power level data from the one or more power sources;
optimize power usage of the one or more power sources;
based on the retrieved power level data, determine whether the one or more power sources are at a predetermined low level of power; and,
based on the determination of whether the one or more power sources are at predetermined low level of power, initiate recharging of the one or more power source;
wherein the operations are performed iteratively to continuously monitor and adapt power distribution and consumption.

9. The implantable physiological interface device of claim 3, wherein the one or more internal components comprise:
a communication module operably coupled to the bioprocessor and configured to be communicably coupled to an external interface and configured to be communicably coupled to another physiological interface device.

10. The implantable physiological interface device of claim 1, further comprising a signal interference mitigation engine operably coupled to the bioprocessor and configured to filter and reduce interference of an input signal.

11. An implantable physiological interface device comprising:
an array of panels, each panel comprising:
a substrate defining opposing first and second sides;
a bioprocessor supported on the first side; and,
an electrode disposed on the second side operatively coupled to the bioprocessor; and,
wherein the substrate of each of the panels joins the substrate of at least one other adjacent panel, such that the array of panels collectively define an outer enclosure having an internal volume configured to house one or more internal components; and,
a data store comprising a program of instructions and operably coupled to the bioprocessor, such that, when the bioprocessor executes the program of instructions, the bioprocessor causes operations to be performed to interface with a physiological environment.

12. The implantable physiological interface device of claim 11, wherein each of the panels in the array lies in a different plane.

13. The implantable physiological interface device of claim 11, further comprising one or more internal components housed within the internal volume of the outer enclosure.

14. The implantable physiological interface device of claim 13, wherein the one or more internal components comprises:
a communication module communicably coupled to the bioprocessor and communicably coupled to an external interface such that the physiological interface device is configured to be remotely monitored via the external interface;
one or more sensors operably coupled to the bioprocessor; and,
wherein, the operations further comprise:
retrieve an input signal from the one or more sensors;
store the input signal in the data store;
based on the input signal, determine whether a predetermined alarm condition has been triggered;
generate output data based on the predetermined alarm condition triggered; and,
transmit the output data to the external interface,
such that the one or more sensors monitor physiological activity and provide diagnostic feedback.

15. The implantable physiological interface device of claim 13, wherein the one or more internal components comprise:
one or more sensors operably coupled to the bioprocessor;
one or more therapeutic components operably coupled to the bioprocessor; and,
wherein, the operations further comprise:
retrieve an input signal from the one or more sensors;
store the input signal in the data store;
based on the input signal, determine whether a predetermined alarm condition has been triggered;
generate a signal representing a therapeutic output based on the predetermined alarm condition triggered;
transmit the signal representing the therapeutic output to the one or more therapeutic components until the predetermined alarm condition is no longer triggered,
wherein receiving the signal representing the therapeutic output activates the one or more therapeutic components to deliver the therapeutic output.

16. The implantable physiological interface device of claim 11, wherein the operations further comprise:
retrieve an input signal from a set of panels that identifies which panels are in contact with a target area of a patient;
analyze the input signal and determine which panels are in contact with the target area and corresponding environmental parameters of the panels in contact with the target area;
based on the analyzed input signal, activate the set of panels in contact with the target area;
monitor changes in contact conditions between the set of panels in contact with the target area and the target area over a predetermined period;
recalibrate the set of panels by deactivating and activating panels in response to monitored changes in contact conditions;
wherein the operations are performed iteratively to continuously adapt an active panel configuration based on real-time contact conditions.

17. The implantable physiological interface device of claim 13, wherein the one or more internal components comprise:
one or more sensors operably coupled to the bioprocessor;
one or more thermal management units operably coupled to the bioprocessor; and,
wherein, the operations further comprise:
retrieve an input signal representing temperature data of the array of panels from the one or more sensors;
analyze the temperature data by comparing the input signal representing temperature data to one or more predetermined thermal thresholds corresponding to a safe operating range;
determine whether the temperature data falls outside the predetermined thermal thresholds;
in response to a determination that the temperature exceeds an upper threshold, activate the one or more thermal management units configured to dissipate heat from the array of panels;
determine whether the temperature returns to within the safe operating range;
wherein the operations are performed iteratively to continuously maintain the temperature within the safe operating range.

18. The implantable physiological interface device of claim 13, wherein the one or more internal components comprise:
a power source module operably coupled to the bioprocessor and configured to monitor and charge one or more power sources; and,
wherein, the operations further comprise:
retrieve power level data from the one or more power sources;
optimize power usage by adjusting at least one of operating frequency, voltage levels, or by deactivating one or more unused components;
based on the retrieved power level data, determine whether the one or more power sources are at predetermined low level of power; and,
based on the determination of whether the one or more power sources are at predetermined low level of power, initiate recharging of the one or more power source;
wherein the operations are performed iteratively to continuously monitor and adapt power distribution and consumption.

19. The implantable physiological interface device of claim 13, wherein the one or more internal components comprise:
a communication module operably coupled to the bioprocessor and configured to be communicably coupled to an external interface and configured to be communicably coupled to another physiological interface device.

20. An implantable physiological interface system comprising:
one or more implantable physiological interface devices configured to communicably couple each other and configured to communicably couple an external interface, the one or more implantable physiological interface devices comprising:
an array of panels, each panel comprising:
a substrate defining opposing first and second sides;
a bioprocessor supported on the first side; and,
an electrode disposed on the second side operatively coupled to the bioprocessor; and,
wherein the substrate of each of the panels joins the substrate of at least one other adjacent panel, such that the array of panels collectively define an outer enclosure having an internal volume configured to house one or more internal components; and,
a data store comprising a program of instructions and operably coupled to the bioprocessor, such that, when the bioprocessor executes the program of instructions, the bioprocessor causes operations to be performed to interface with a physiological environment.
